(19) United States
(12) Patent Application Publication
(10) Pub. No.: US 2009/0152148 A1
(43) Pub. Date: Jun. 18, 2009

(54) STERILE PACKAGING OF A MEDICAL ARTICLE

(76) Inventors: Simon Scheurer, Bern (CH); Reto Aeschlimann, Aefligen (CH)

Correspondence Address: Dorsey & Whitney LLP, IP Department, ATTN: Disetronic Licensing AG, 50 South Sixth Street, Suite 1500, Minneapolis, MN, 55402-1498, US

(21) Appl. No.: 12/333,634

(22) Filed: Dec. 12, 2008

Related U.S. Application Data
(63) Continuation of application No. PCT/CH2007/000076, filed on Feb. 13, 2007.

(30) Foreign Application Priority Data
Jun. 13, 2006 (CH) 956/06

(51) Int. Cl. A61B 19/02

(52) U.S. Cl. 206/365

(57) ABSTRACT

A container for containing a medical device, the container including a shell with a flange area, a packaging chamber formed by the shell, a cover removably connected to the shell in the flange area, a disposal chamber formed by shell, and a latch for securing the medical device in the container. The invention encompasses a method of using the container for containing the medical device before and after use of the device. In one embodiment, the disposal chamber is adjacent to the shell and includes a latch for connecting to the medical device and, in another embodiment, the packaging chamber and disposal chamber are the same and the container further includes a cover foldably connected to the shell and a latch for locking the cover to the shell.

### CROSS-REFERENCE TO RELATED APPLICATIONS

This application is a continuation of International Patent Application No. PCT/CH2007/00076, filed on Feb. 13, 2007, which claims priority to Swiss Application No. 956/06, filed on Jun. 13, 2006, the entire contents of both of which are incorporated herein by reference.

### BACKGROUND

The present invention relates to medical devices or articles, and to methods of making, using and packaging such devices. More particularly, it relates to packages, containers and/or receptacles for medical devices, and to methods of making and using such containers, packages and/or receptacles, including for initially containing such devices and/or disposal of such devices subsequent to their use. More particularly, the present invention relates to a container and a method for disposal of a medical article having a needle or blade like element for puncturing or cutting the body of a patient, to an application or use of the method, as well as to sterile packaging of a medical article.

Sterile medical articles, which are used to puncture or cut the body of a patient, e.g. infusion needles, scalpels, ports or infusion sets for insulin pumps, today mostly are provided as disposables in sterile packaging. This development has lead to a considerable simplification in the logistics for providing such articles, e.g. in the field of ambulant medicine. However, the safe disposal of such articles, which after use are contaminated, constitutes a problem that until now has not satisfyingly been addressed. In recent years this problem is worse due to the spread of diseases such as AIDS.

While an unintended coming into contact and harming of a person with a sterile medical article upon a removal of the article from packaging is painful and, in most cases, renders the article unusable for further use, it normally has no sanitary consequences for the harmed person. However, in case of an injury caused by a contaminated article there is the risk that dangerous diseases are transferred. Accordingly, it is of utmost importance to organize the handling and disposing process in such a manner that the risk of harm from the contaminated article is as little as possible.

According to the prior art, e.g. in doctor's practices, after use infusion needles are collected in specific collecting containers and then are jointly disposed of. This, however, has the disadvantage that the contaminated needles stay for a longer period undisposed, so that there still is a risk of infection, e.g. via the air or via liquids that might leak from the collecting container. Furthermore, the contaminated needles need to be handled a second time for disposal, which again generates a risk of injury.

From U.S. Pat. Nos. 5,293,993 and 4,979,616 disposal containers for used syringes are known, in which the syringes including a needle are introduced and by irreversible closing of the container are permanently protected against coming into contact with persons, so that they can be disposed of in due course. Such solutions, however, have the disadvantage that the disposal containers need to be separately sourced and managed, which is cost intensive and generates the risk that temporarily there may be no disposal containers available even though there are contaminated syringes that need to be disposed of.

U.S. Pat. No. 6,235,006 discloses catheters with a protective housing arranged around the catheter. After use, a needle can be pulled into the housing and be irreversibly latched in a protected position, so that disposal in normal household waste is possible without any problem. Such a solution, however, is cost intensive and limited to applications or devices wherein such an arrangement of a protective housing is possible from a technical point of view, as well does not interfere with the intended use of the article.

### SUMMARY

It is an object of the present invention to provide packaging or containing methods and products which do not have the disadvantages of the prior art or at least partially avoid them.

In one embodiment, the present invention comprises a container for containing a medical device, the container comprising a shell with a flange area, a packaging chamber formed by the shell, a cover removably connected to the shell in the flange area, a disposal chamber formed by shell, and a latch for securing the medical device in the container. The invention encompasses the a method of using the container for containing the medical device before and after use of the device. In one embodiment, the disposal chamber is adjacent to the shell and comprises a latch for connecting to the medical device and, in another embodiment, the packaging chamber and disposal chamber are the same and the container further comprises a cover foldably connected to the shell and a latch for locking the cover to the shell.

One aspect of the present invention relates to a method for the disposal of a medical article having a sharp, pointed needle or blade-like element for puncturing or cutting the body of a patient, e.g. an infusion needle, a scalpel, a port or an infusion set for an insulin pump. In one embodiment of the method, the article or at least the part of the article which comprises the needle or blade-like element, after removal from the packaging and the intended use, is introduced into a disposal chamber that is provided by the packaging, and at the same time or subsequently is secured against a removal from the disposal chamber with a lock or securing means provided by the packaging. Thus, an unintended contact with the contaminated needle or blade like element is reliably obviated.

The present invention provides that a medical article or a part thereof which provides a needle or blade like element, can immediately, after use, be disposed of in a safe manner with the household waste by using the original packaging. This results in the advantage that complicated interim storage is avoided and the contaminated needle or blade like element needs to be handled only once, resulting in a reduction of the risk of injury. Furthermore, it results in the advantage that the sales or supply packaging also forms the disposal packaging. Thus, there is automatically available a disposal container adapted to the article without the need to separately provide such a container. The safety of the disposal process is increased and logistics are facilitated, since for providing and disposing of the article less packaging waste is generated, so that savings in the providing and disposal costs can be achieved.

The present invention relates to packaging of an infusion set for insulin, in which the infusion set is arranged inside a sterile packaging chamber formed by a packaging shell and closed by a sealing cover. The sealing cover is sealed onto a flange area of the packaging shell and for opening the packaging chamber can be separated from the packaging shell along the sealing connection. In one embodiment, adjacent to the packaging chamber the packaging shell provides a separate disposal chamber with latching noses, inside which the handle part of the infusion set that carries a guide needle, which handle part is removed after the application of the infusion set to the patient, together with the guide needle, can be arranged and by the latching noses can be latched and held in a position in which an unintended coming into contact of persons with the contaminated guide needle is not possible. Due to the fact that, immediately after use, the handle part with the guide needle can safely be contained in the original packaging and disposed of with normal household waste, the risk of injury is reduced, as are costs associated with production and disposal.

In a preferred embodiment of the method according to the present invention, the medical device or article or the part thereof which provides a needle or blade-like element, is, after its intended use, re-introduced in a chamber in the package in which it was arranged for its provision. Thus, in this case, a disposal chamber according to the present invention claims corresponds to the original packaging chamber. In an alternative preferred embodiment of the method according to the present invention, the article or the part of the article which provides the needle or blade-like element, is, after its intended use, introduced in a chamber in the packaging in which it was not arranged for its provision. Thus, in this case, a disposal chamber according to the present invention is a different chamber than the original packaging chamber. Depending on the article and the packaging variant, one of these embodiments might be more preferable than the other embodiment.

In a further preferred embodiment of the method according to the present invention, the disposal chamber, for securing the article or the part of the article that provides the needle or blade like element against a removal, is closed with a safety cover provided by and/or integral with the packaging. In some preferred embodiments, the closing of the disposal chamber takes place in an irreversible manner, so that opening or reopening it is not possible or is only possible upon use of force or destruction of elements of the packaging and/or of the article. In another preferred embodiment of the method, for securing the article or the part of the article that provides the sharp, needle or blade-like element against a removal from the disposal chamber, the article is, during or after its introduction into the disposal chamber in a position in which an unintended coming into contact of persons with it is impossible. In this embodiment, the chamber is latched with one or several latching elements provided by the packaging, and it may be preferred that the latching takes place in an irreversible manner, so that releasing the latching is not possible or only upon use of force or destruction of elements of the packaging and/or of the article. Depending on the article and the packaging variant, one of these embodiments might be more preferable than the other embodiment, or a combination of both embodiments might be preferable.

Another aspect of the present invention relates to an application or use of the method according to present invention for the disposal of infusion sets or parts of infusion sets for the subcutaneous feeding of liquid drugs, e.g. of insulin, into the body of a person. In such applications, the advantages of the present invention clearly become evident.

Another aspect of the present invention relates to a sterile package or container for a medical article for puncturing or cutting in the body of a patient which is suitable for being used in the method according to the present invention. The sterile package or container (which also may be referred to as packaging) comprises a sterile packed medical article having a needle or blade like element for puncturing or cutting in the body of a patient, which is arranged in a packaging chamber formed by the container. This packaging chamber can be opened without any tools by hand, so that the article, as the case may be with a further sterile cover surrounding it, can be taken out of the packaging chamber. After the removal of the medical article from the packaging chamber and its intended use, it can be introduced into a disposal chamber provided by the container and be secured therein by securing means provided by the container so that an unintended contact with the contaminated needle or blade like element is not possible. After the securing accidental or intentional contact with the dangerous part of the device is no longer possible, or at least no longer possible without purposeful manipulation. In some preferred embodiments, the parts of the packaging surrounding the contaminated medical device, needle or blade like element may be designed to resist a piercing or cutting through the same under normal disposal conditions.

By providing medical articles having a needle or blade like element for puncturing or cutting in the body of a patient inside such a sterile packaging or container there is automatically provided a disposal container adapted to the article, without the need to separately take care for the provision of such. The medical article or the part thereof that provides the needle or blade like element can, directly after the intended use, by use of the original packaging be disposed of with the normal household waste. Thus, potentially problematic interim storing is avoided and the contaminated needle or blade like element needs to be handled only once, so that the risk of injury can be reduced to a minimum. Additionally, there is the advantage that less packaging waste is generated, since the packaging used for sales and provision of the article also serves for the disposal of it. Costs for the provision and disposal can be reduced.

In a preferred embodiment of the sterile packaging in accordance with the present invention, the packaging comprises securing means designed in such a manner that the securing of the article or the part of the article which provides the needle or blade like element, which is introduced into the disposal chamber, is irreversible. Thus, a maximum of safety against a later contact with the contaminated needle or blade like element is achieved.

In another preferred embodiment of the sterile packaging in accordance with the present invention, the packaging comprises a base part, which forms the packaging chamber, and a cover part, which seals the packaging, wherein the cover part can, without tools, be partially or entirely separated from the base part or destroyed for permitting removal of the article from the packaging chamber. Thus, the cover part can be destroyed or separated from the base part at least to such an extent that removal of the sterile article out of the packaging chamber is possible without difficulty. In some embodiments, the cover part is completely separated from the base part and, in other embodiments, stays undetachably attached at the base part, which provides the advantage that it does not need to separately be disposed.

In some preferred embodiments, the present invention comprises a disposal chamber and securing means formed by the base part, e.g. a one piece design of these elements.

In some preferred embodiments wherein packaging in accordance with the present invention comprises a base part and a cover part, the connection between the cover part and the base part comprises one or several predetermined breaking points, weak points or a line or lines of weakness whereby the cover can be separated from the base part without tools. In some embodiments, the separation destroys the connection. Such packaging has the advantage that opened packages can immediately and clearly be distinguished from unopened packages, which for sterile packaging is of important.

In some embodiments, the cover part is sealed onto a flange portion provided by the base part.

In some embodiments, the base part is formed from a thermoplastic material by injection molding or through thermal forming of a thermoplastic plastic film. Such base parts are cheap and generate relative little waste.

In some embodiments, the cover part is formed by a medical paper, a paper like fleece or fabric (e.g. that available from the company Du Pont under the name “Tyvek”), an aluminum film or other suitable material.

In another preferred embodiment of the sterile packaging in accordance with the present invention, the disposal chamber is formed by the original packaging chamber. In another preferred embodiment, the disposal chamber is formed separately from the packaging chamber. Depending on the packaged article and the design of the packaging, one of these embodiments might be more preferable than the other embodiment.

In another preferred embodiment of sterile packaging in accordance with the present invention, the securing means comprises a securing cover by which the disposal chamber, after the introduction of the article or of the part of the article providing the needle or blade like element, can be closed, in some preferred embodiments irreversibly. Thus, reopening is precluded or possible only with use of destructive force. In some embodiments, the securing means is integral with the base.

In another preferred embodiment in accordance with the present invention, the securing means comprises one or several latching or locking elements, by which the article or dangerous part of the article can be latched in a position in the disposal chamber in which the contaminated needle or blade like element is not accessible. In some preferred embodiments, the connection of the latch or lok elements is irreversible. In embodiments in which the packaging comprises a base part and a cover part, the latching elements may be formed integrally with the base part.

In another preferred embodiment of sterile packaging in accordance with the present invention, the medical device or article is a disposable, e.g. an infusion set for the subcutaneous feeding of a liquid drug, e.g. insulin, into the body of a patient.

In some preferred embodiments, the packaging or container in accordance with the present invention is adapted to contain a single medical device or article, in other preferred embodiments, it can contain several articles, wherein it comprises for each article an initial packaging chamber that separately can be opened, a separate disposal chamber, each having separate securing means. While in many embodiments the present invention comprises containing or packing items in a sterile condition, the structures, function and principles of the invention may be applied or used for containing, packing, vending, shipping, storing and/or disposing of items, including items other than medical devices, in a non-sterile condition as well.

### BRIEF DESCRIPTION OF THE DRAWINGS

FIG. 1 is a perspective top view of one embodiment of a sterile package or container according to the present invention, containing an infusion set in an original state;

FIG. 2 is a perspective top view of the sterile packaging of FIG. 1, after the removal of a sealing cover from the packaging shell;

FIG. 3 is a top view of the empty packaging shell of FIG. 2;

FIG. 4 is a vertical section through the empty packaging shell along line A-A of FIG. 3;

FIG. 5 is a perspective view of the packaging shell of FIG. 3 after a latching a used handle part carrying a guide needle in a disposal chamber;

FIG. 6 is a vertical section along line B-B of FIG. 5;

FIG. 7 is a perspective top view of a second embodiment of a package or container according to the present invention, containing an infusion set in an original state;

FIG. 8 is a side view of the package of FIG. 7;

FIG. 9 is a perspective top view of the package of FIG. 7 after the swinging-open of a securing cover;

FIG. 10 is a perspective top view of the package of FIG. 9 after the removal of a sealing cover from the package shell;

FIG. 11 is a perspective top view of the package shell of FIG. 7 after the introduction of a used medical device into the disposal chamber;

FIG. 12 is a perspective top view of the package shell of FIG. 11 after the closing and latching of the securing cover;

FIG. 13 is a side view of the package shell of FIG. 12; and

FIG. 14 is a detailed view, designated X in FIG. 13, partly in section.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding, including sonic welding. Generally, unless otherwise indicated, the materials for making the invention and/or its components may be selected from appropriate materials such as metal, fabrics, metallic alloys, ceramics, plastics, etc.

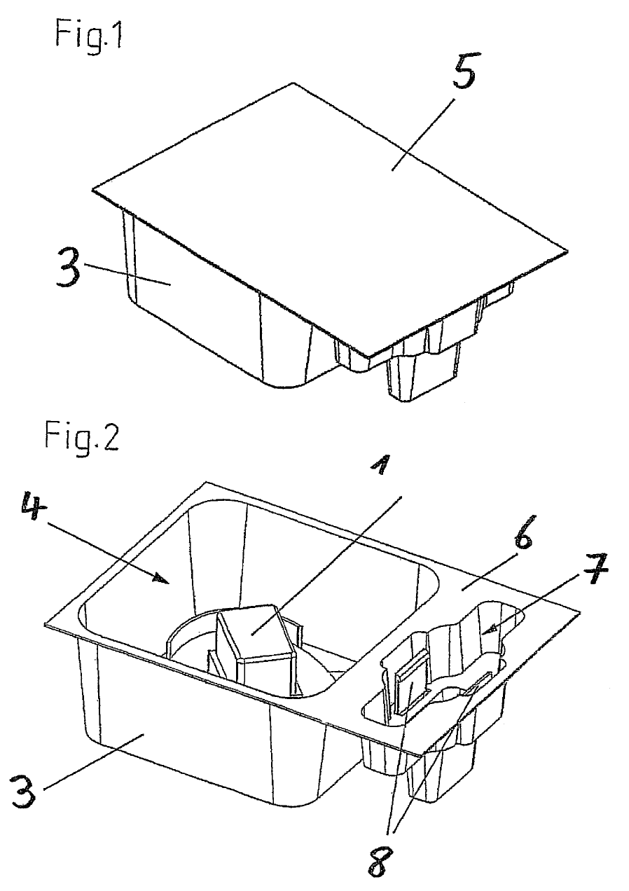

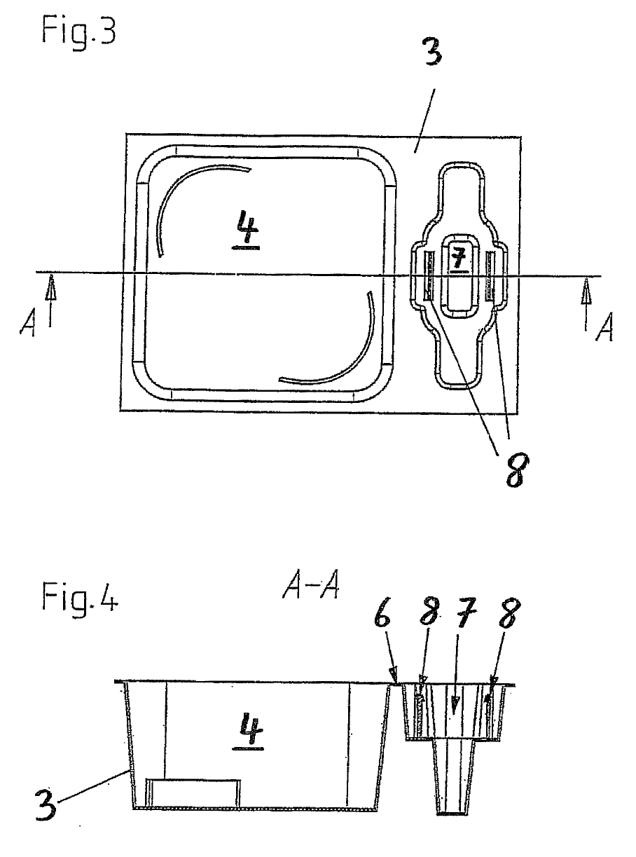

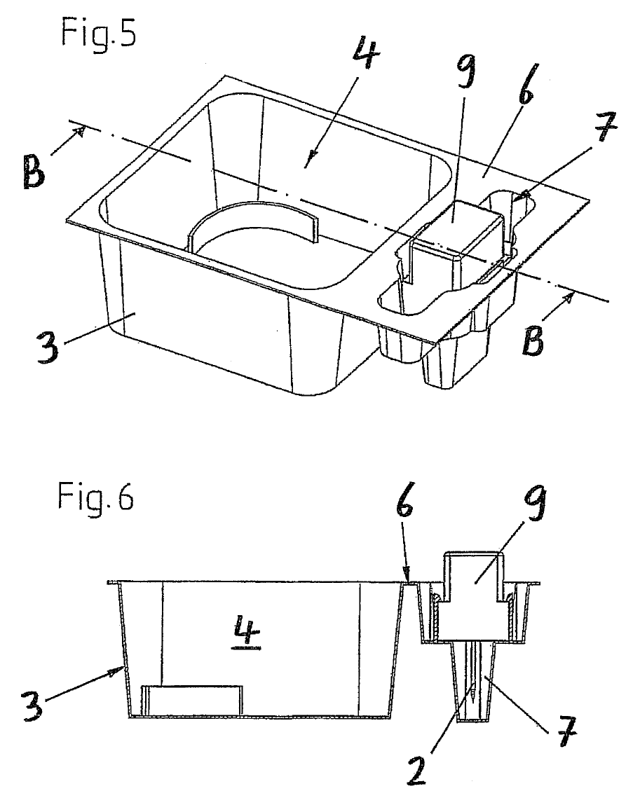

One preferred embodiment of the sterile packaging or container according to the present invention is shown in perspective in FIGS. 1 and 2. The package comprises a shell 3 and sealing cover 5. FIG. 1 shows it opened in an original state, and FIG. 2 shows it after opening via removal of the sealing cover 5. FIGS. 3-6 show the packaging shell 3 in an empty state, in a top view (FIG. 3) and in a vertical section along line A-A in FIG. 3 (FIG. 4). They also show the shell in a state ready for disposal with a handle part 9 and/or contaminated guide needle 2 secured in the packaging shell 3, namely in a perspective top view of the packaging shell (FIG. 5) and in a vertical section along line B-B in FIG. 5 (FIG. 6).

As becomes clear when looking at the Figures in combination, packaging in accordance with the present invention (which also may be thought of and/or referred to as a receptacle, container, package or synonymous terms) consists of a container formed by a packaging shell 3 and a sealing cover 5. An infusion set 1 for insulin is packed or contained, in a sterile condition, inside the container. The packaging shell 3 forms a packaging chamber 4, in which the infusion set 1 is received and which, in the original state, is closed by the sealing cover 5. For this purpose, the sealing cover 5 is sealed onto a flange area 6 formed by the packaging shell 3 in such a manner that, for opening the packaging, it can be detached from the flange area 6 by hand. When detached or broken, the sealing connection with the flange area 6 is irreversibly destroyed.

Adjacent to the packaging chamber 4 the packaging shell 3 forms a separate disposal chamber 7, which in the original state is closed by the sealing cover 5. In the disposal chamber 7 there are two latching noses 8 formed as one piece or integrally with the packaging shell 3. The latching noses provide for securing or latching the handle part 9 of the infusion set 1, which carries a guide needle, in the disposal chamber 7 in a position in which the needle 2 is not accessible. This situation is shown in the FIGS. 5 and 6. A breaking of the latching between the latching noses 8 and the handle part 9 without tools is not possible or only possible under use of force, so that an unintended contact and/or harming of persons with the contaminated guide needle 2 is obviated. In the present embodiment, the packaging shell 3 is produced from a thermoplastic plastic material by injection molding, with a thickness of about 1.0 mm, and the sealing cover 5 consists of a membrane, film, fleece or other suitable material which, on one side, is coated with a sealing glue.

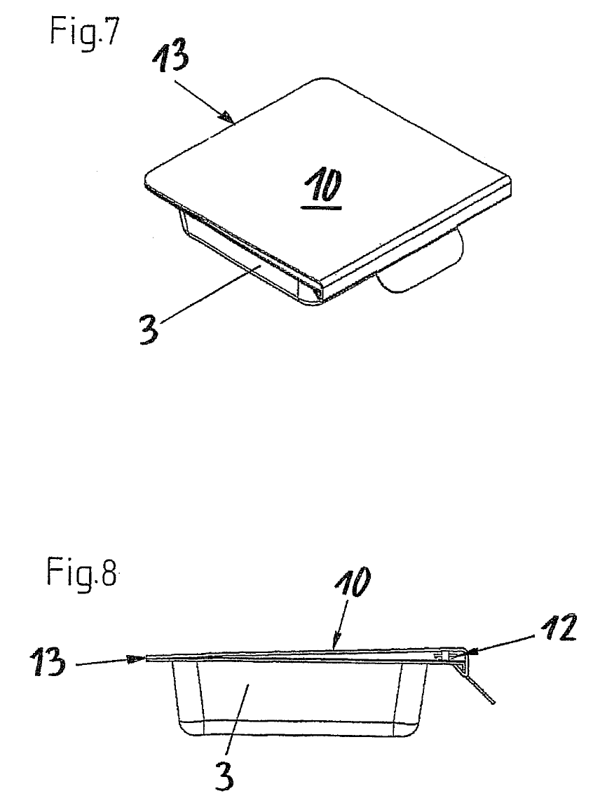

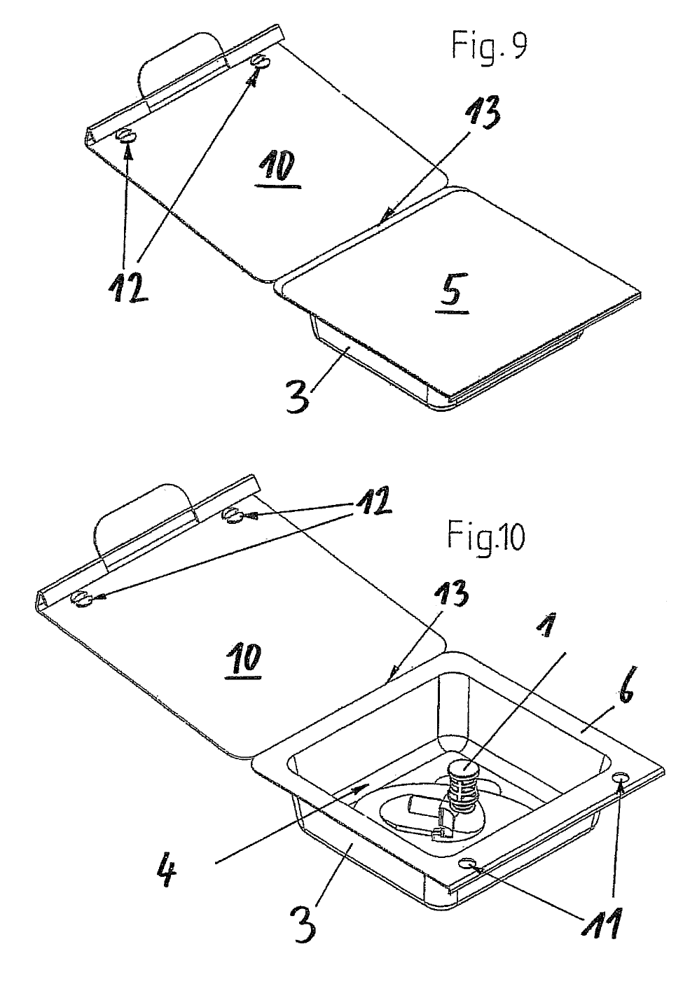

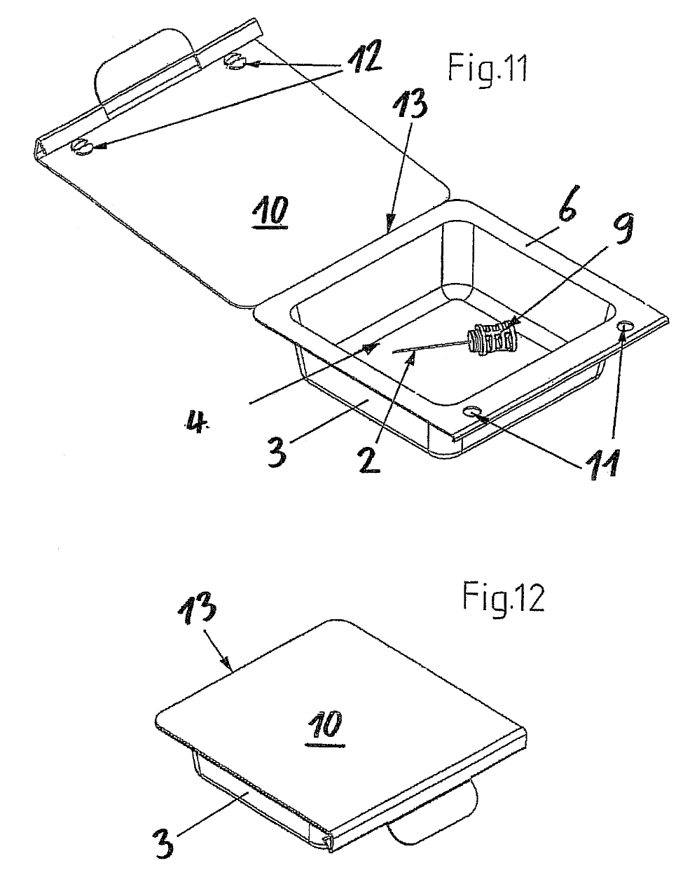

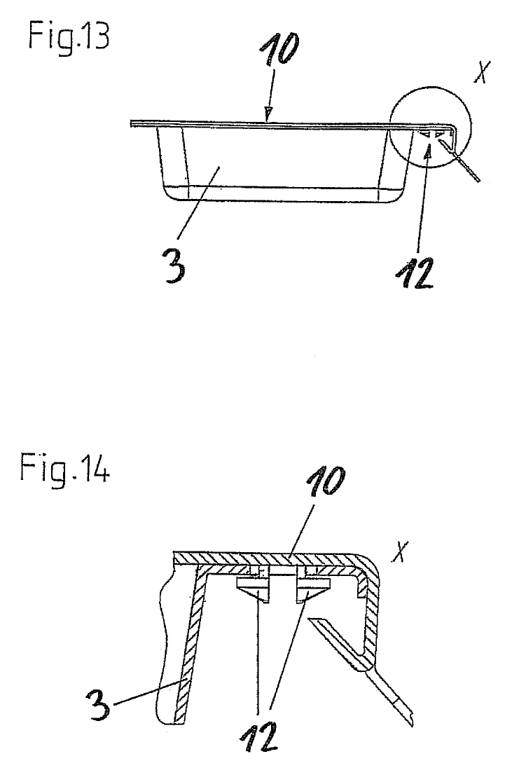

Another preferred embodiment of the packaging or container according to the present invention is shown in FIGS. 7 to 10. The package is shown complete and not opened in the original state in FIGS. 7 to 9 and in an opened state in FIG. 10. FIGS. 7 and 8 show the packaging with a closed protective cover 10, in a perspective top view (FIG. 7) and in a side view (FIG. 8). FIG. 9 is a perspective top view showing the package with the protective cover 10 open. FIG. 10 shows it after the removal of the sealing cover 5 from the packaging shell 3. FIG. 11 shows a perspective top view of the packaging shell 3 after the introduction of a handle part 9 carrying a contaminated guide needle 2 into a packaging chamber 4, which now serves as disposal chamber 4. FIGS. 12 and 13 show the packaging shell 3 which contains the handle part 9 with the contaminated guide needle 2 closed by the protective cover 10, which now serves as securing cover, with the package in a state ready for disposal, in a perspective top view (FIG. 12) and in a side view (FIG. 13).

In this embodiment, the packaging consists of a package or container containing an infusion set 1 for delivery insulin which in the packaging in a sterile manner (FIG. 10). The package comprises a packaging shell 3 and a sealing cover 5. In the sales or purchase state, the sealing cover 5 is protected against damage by a protective cover 10 that can be folded away, which protective cover is made in one piece with the packaging shell 3 and is interconnected therewith along a hinge band 13 (FIGS. 7 and 8) or other suitable structure. The packaging shell 3 forms a packaging chamber 4, in which the infusion set 1 is received and which, in its original state is closed by the sealing cover 5. The sealing cover 5, which is only accessible after folding away of the protective cover 10 (FIG. 9), is sealed onto a flange area 6 provided by the packaging shell 3 in such a manner that, for opening of the package, it can be removed by hand from the flange area 6. By doing so, the sealing connection with the flange area 6 is irreversibly destroyed. Also, latching openings 11 are exposed or uncovered for a later latching of latching protrusions 12 arranged at the protective cover 10. This situation is shown in FIG. 10. In this embodiment, the packaging shell 3 is formed by a thermally formed PET-film having a thickness of 0.7 mm. As in the previous embodiment, the sealing cover 5 again consists of a fleece or other suitable material which is coated with a sealing glue on an appropriate surface thereof.

In contrast to the previous embodiment, the packaging shell 3 depicted in FIGS. 7-10 does not form an additional disposal chamber. The chamber 4 which served as packaging chamber, after the removal and application of the originally therein arranged infusion set 1, serves as disposal chamber for receiving the handle part 9 with the contaminated guide needle 2, which handle part has been removed from the applied infusion set. After the handle part has been introduced into the disposal chamber 4 (see FIG. 11), the protective cover 10, which now serves as securing cover 10 is closed and its latching protrusions, noses or elements 12 are received in and latched or locked inside the latching openings 11. The disposal chamber 4 is reliably closed and an unintended contact and/or harming of persons by the contaminated guide needle 2 arranged therein is obviated. This situation is shown in FIGS. 12-14, wherein the latching of the latching noses 12 in the complementary, dedicated latching opening 11 is shown in partial section in FIG. 14, a detail X taken from FIG. 13.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A method for disposing of a medical article comprising a needle or blade-like element for puncturing or cutting the body of a patient after the removal of said article from a sterile packaging, the method comprising the steps of:
a) after the intended use the article, introducing the article into a disposal chamber formed by the packaging; and
b) securing the article against removal from the disposal chamber by using securing means provided by the packaging, such that unintended with the contaminated needle or blade-like element is impossible.

2. The method according to claim 1, wherein before the intended use of the article, the disposal chamber served as a packaging chamber.

3. The method according to claim 1, wherein before the intended use of the article, the disposal chamber has not served as a packaging chamber.

4. The method according to claim 1, wherein the article is locked in the disposal chamber.

5. The method according to claim 1, wherein the disposal chamber is closeable by a securing cover.

6. The method according to claim 4, wherein the securing cover is latched by at least one latching element integral with the packaging in a position in which an unintended contact with the contaminated needle or blade like element is impossible.

7. The method according to claim 1, wherein the medical article is an infusion sets or part of an infusion set.

8. A container for containing a medical device, the container comprising:
a shell with a flange area;
a packaging chamber formed by the shell;
a cover removably connected to the shell in the flange area;
a disposal chamber formed by shell; and
a latch for securing the medical device in the container.

9. The container according to claim 8, the disposal chamber is adjacent to the packaging chamber and comprises a latch for connecting to the medical device.

10. The container according to claim 8, wherein the packaging chamber and disposal chamber are the same and the container further comprises a cover foldably connected to the shell and a latch for locking the cover to the shell.

11. A sterile packaging for a medical article having a needle or blade like element for puncturing or cutting the body of a patient, comprising a packaging chamber formed by the packaging, which can be opened without any tools, for enabling removal of the article from the packaging chamber, wherein after the removal and the intended use of the article, the article is introduced into a disposal chamber formed by the packaging and is secured therein by securing means formed by the packaging such that an unintended contact of persons with the needle or blade like element is prevented.

12. The sterile packaging according to claim 11, wherein the securing means is designed such that the securing of the article in the disposal chamber is irreversible.

13. The sterile packaging according to claim 12, wherein the packaging chamber is a sterile chamber.

14. The sterile packaging according to claim 11, further comprising a base part, which forms the packaging chamber, and a cover part, which closes the packaging chamber, wherein the cover part is partly or completely separable from the base part or can be destroyed without tools for enabling the removal of the article from the packaging chamber.

15. The sterile packaging according to claim 14, wherein the disposal chamber and the securing means are formed by the base part.

16. The sterile packaging according to claim 15, wherein the connection between the cover part and the base part is designed such that the cover part, for enabling of a removal of the article, can be separated from the base part along predetermined breaking points without tools and the connection in the area of the predetermined breaking points is irreversibly destroyed.

17. The sterile packaging according to claim 16, wherein the cover part is sealed onto a flange area formed by the base part.

18. The sterile packaging according to claim 17, wherein the base part is one of formed of a thermoplastic by injection molding or formed of a thermoplastic plastic film by thermal forming.

19. The sterile packaging according to claim 18, wherein the cover part is formed by one of a medical paper, a paper like fleece or an aluminum film.

20. The sterile packaging according to claim 11, wherein the disposal chamber is formed by the packaging chamber.

21. The sterile packaging according to claim 11, wherein the disposal chamber is formed separately from the packaging chamber.

22. The sterile packaging according to claim 11, wherein the securing means comprises a securing cover by which the disposal chamber can be closed irreversibly.

23. The sterile packaging according to claim 22, wherein the securing cover is formed in one piece with the base part.

24. The sterile packaging according to claim 23, wherein the securing means comprises at least one latching element for irreversibly latching the article or the part of the article that provides the needle or blade like element into the disposal chamber whereby an unintended contact with the article or the part of the article that provides the needle or blade like element is not possible.

25. The sterile packaging according to claim 24, wherein the at least one latching element is formed in one piece with the base part.

26. The sterile packaging according to claim 11, wherein the article is a disposable infusion set for the subcutaneous delivery of insulin into the body of a patient.

27. The sterile packaging according to claim 26, wherein the packaging contains one article.

28. The sterile packaging according to claim 26, wherein the packaging contains more than one article and comprises a disposal chamber and securing means for each of said more than one article.

29. The sterile packaging according to claim 28, wherein each article is in a packaging chamber which can separately be opened.
